(19) United States
(12) Patent Application Publication
(10) Pub. No.: US 2010/0159576 A1
(43) Pub. Date: Jun. 24, 2010

(54) BIOCHIP AND BIOMATERIAL DETECTION APPARATUS

(75) Inventors: Hyun Woo SONG, Daejeon (KR); Dae-Sik Lee, Daejeon (KR); Hyeon-Bong Pyo, Daejeon (KR); Kwang Hyo Chung, Daejeon (KR); Moon Youn Jung, Daejeon (KR)

Correspondence Address: AMPACC Law Group, 3500 188th Street S.W., Suite 103, Lynnwood, WA, 98037, US

(73) Assignee: Electronics and Telecommunications Research Institute, Daejeon (KR)

(21) Appl. No.: 12/643,969

(22) Filed: Dec. 21, 2009

(30) Foreign Application Priority Data
Dec. 22, 2008 (KR) 10-2008-0130959

(51) Int. Cl. C12M 1/34; G01N 21/64

(52) U.S. Cl. 435/288.7; 422/82.08

(57) ABSTRACT

Provided are a biochip and a biomaterial detection apparatus. The biochip includes a substrate, a metal layer, and a dielectric layer. The substrate includes a surface having a plurality of acute parts which are formed by first and second inclined planes. The metal layer is formed on at least one of the first and second inclined planes. The dielectric layer is formed on the metal layer, and capture molecules specifically binding to target molecules which are marked with a fluorescent substance are immobilized to a surface of the dielectric layer.

### CROSS-REFERENCE TO RELATED APPLICATIONS

This U.S. non-provisional patent application claims priority under 35 U.S.C. §119 of Korean Patent Application No. 10-2008-0130959, filed on Dec. 22, 2008, the entire contents of which are hereby incorporated by reference.

### BACKGROUND OF THE INVENTION

The present invention disclosed herein relates to a biochip and a biomaterial detection apparatus, and more particularly, to a biomaterials detection apparatus using surface plasmon resonance.

A biomaterial detection apparatus (i.e., biosensor) is a device that may detect an optical signal or an electrical signal which is changed according to selective reaction and binding between a biological receptor having a recognition function for specific biomaterials and an analyte to be analyzed. That is, the biosensor may check the existence of biomaterials or qualitatively or quantitatively analyze the biomaterials. As the biological receptor (i.e., sensing materials), enzymes, antibodies and DNA that may selectively react and bind to specific materials are used. By using various physico-chemical methods such as the change of an electrical signal based on the presence of an analyte and the change of an optical signal based on a chemical reaction between a receptor and an analyte as a signal detection method, biomaterials are detected and analyzed.

In the case of an optical biosensor using the change of an optical signal, much research is actively being made on biosensors using optical methods such as surface plasmon biosensors, total internal reflection ellipsometry biosensors and waveguide biosensors.

### SUMMARY OF THE INVENTION

The present invention provides a biochip, which more easily excites a surface plasmon, thereby improving the sensing efficiency of a fluorescent signal for the analysis of biomaterials.

The present invention also provides a biomaterial detection apparatus, which more easily excites a surface plasmon, thereby improving the sensing efficiency of a fluorescent signal for the analysis of biomaterials.

The object of the present invention is not limited to the aforesaid, but other objects not described herein will be clearly understood by those skilled in the art from descriptions below.

Embodiments of the present invention provide a biochip including: a substrate including a surface which has a plurality of acute parts formed by first and second inclined planes; a metal layer on at least one of the first and second inclined planes; and a dielectric layer on the metal layer, in which capture molecules, specifically binding to target molecules which are marked with a fluorescent substance, are immobilized to a surface of the dielectric layer.

In other embodiments of the present invention, a biomaterial detection apparatus includes: a substrate including a surface which has a plurality of acute pails formed by first and second inclined planes; a metal layer on at least one of the first and second inclined planes; a dielectric layer on the metal layer, in which capture molecules, specifically binding to target molecules which are marked with a fluorescent substance, are immobilized to a surface of the dielectric layer; a light source unit irradiating an excitation light at a predetermined angle for the first or second inclined plane of the substrate; and a detection unit detecting an emission light which is emitted from the fluorescent substance which is immobilized by specifically binding between the capture molecules and the target molecules, in one of the first and second inclined planes of the substrate.

### BRIEF DESCRIPTION OF THE FIGURES

The accompanying figures are included to provide a further understanding of the present invention, and are incorporated in and constitute a part of this specification. The drawings illustrate exemplary embodiments of the present invention and, together with the description, serve to explain principles of the present invention. In the figures:

FIG. 1 is a diagram illustrating a biochip according to an embodiment of the present invention;

FIG. 2 is a diagram illustrating a biochip according to another embodiment of the present invention;

FIGS. 3A through 3C are diagrams illustrating a method of fabricating a biochip according to an embodiment of the present invention;

FIG. 4 is a diagram illustrating a biomaterial detection apparatus according to an embodiment of the present invention;

FIG. 5 is a graph illustrating the change of a reflection rate based on an incident angle of an excitation light; and

FIG. 6 is a diagram illustrating a biomaterial detection apparatus according to another embodiment of the present invention.

### DETAILED DESCRIPTION OF PREFERRED EMBODIMENTS

Preferred embodiments of the present invention will be described below in more detail with reference to the accompanying drawings. The present invention may, however, be embodied in different forms and should not be construed as limited to the embodiments set forth herein. Rather, these embodiments are provided so that this disclosure will be thorough and complete, and will fully convey the scope of the present invention to those skilled in the art. Further, the present invention is only defined by scopes of claims. Therefore, in some embodiments, well-known processes, device structures, and technologies will not be described in detail to avoid ambiguousness of the present invention. An embodiment described and exemplified herein includes a complementary embodiment thereof. Like reference numerals refer to like elements throughout.

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises” and/or “comprising,” when used iii this specification, specify the presence of stated features, integers, steps, operations, elements, and/or components, but do not preclude the presence or addition of one or more other features, integers, steps, operations, elements, components, and/or groups thereof.

Additionally, the embodiment in the detailed description will be described with sectional views as ideal exemplary views and/or plan views of the present invention. In the figures, the dimensions of layers and regions are exaggerated for clarity of illustration. Therefore, areas exemplified in the drawings have general properties, and are used to illustrate a specific shape of the region of a device. Thus, this should not be construed as limited to the scope of the present invention.

In specification, target molecules are biomolecules which show specific nature, and may be interpreted as the same meaning as a body for analysis or analytes. In embodiments of the present invention, the target molecules correspond to antigens.

In specification, capture molecules are biomolecules that specifically binds to the target molecules, and may be interpreted as the same meaning as probe molecules, a receptor or an acceptor. In embodiments of the present invention, the capture molecules correspond to capture antibodies.

In embodiments of the present invention, moreover, a sandwich immuno-assay is used for detecting biomaterials. The sandwich immuno-assay is a method that specifically binds target molecules to sensing molecules and specifically binds the target molecules bound to the sensing molecules to capture molecules to form the conjugate of capture molecules-target molecules-sensing molecules structure, thereby detecting biomaterials.

Hereinafter, it will be described about an exemplary embodiment of the present invention in conjunction with the accompanying drawings.

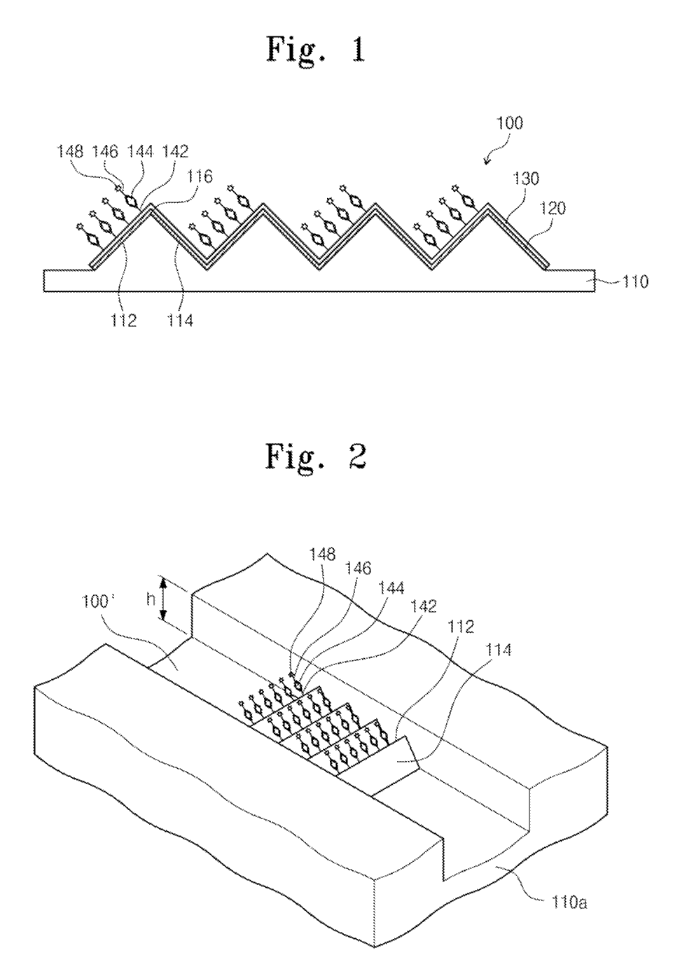

FIG. 1 is a diagram illustrating a biochip according to an embodiment of the present invention.

Referring to FIG. 1, a biochip 100 according to an embodiment of the present invention includes a substrate 110, a metal layer 120, a dielectric layer 130, and capture molecules 142 specifically binding to target molecules 144.

The substrate 110 may be formed of a material that may transmit or reflect light. For example, the substrate 110 may be a plastic substrate, a glass substrate or a silicon substrate. Moreover, the substrate 110 may be formed of a polymer such as polydimethylsiloxane (PDMS), polymethylmethacrylate (PMMA), polycarbonate (PC), cyclic olefin copolymer (COC), polyamide (PA), polyethylene (PE), polypropylene (PP), polyphenylene ether (PPE), polystyrene (PS), polyoxymethyleue (POM), polyetheretherketone (PEEK), polytetrafluoroethylene (PTFE), polyvinylchloride (PVC), polyvinylideuefluoride (PVDF), polybutyleneterephthalate (PBT), fluorinated ethyleuepropylene (FEP) and perfluoralkoxyalkane (PFA).

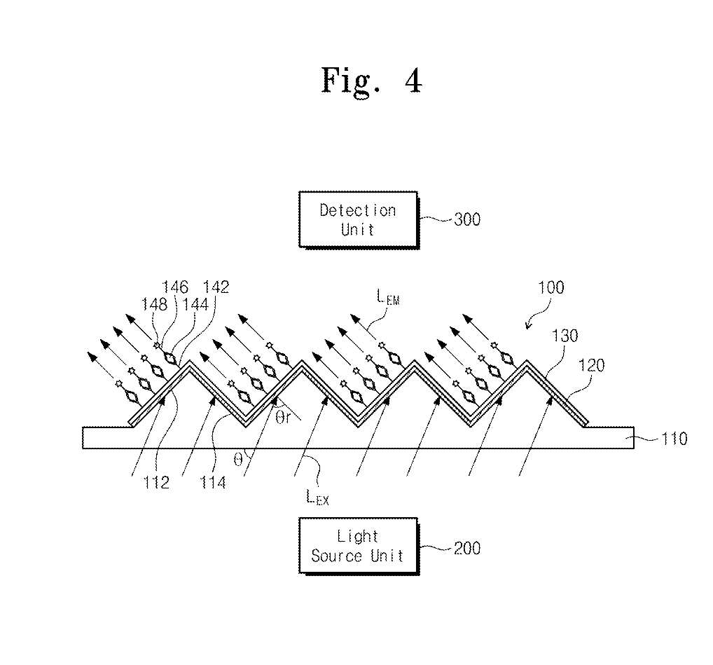

The substrate 110 includes a wedge shape of an upper surface at a predetermined region. Specifically, acute parts 116 formed by first and second inclined planes 112 and 114 may be formed at the upper surface of the substrate 110, and a plurality of acute parts 116 may be formed at the upper surface of the substrate 110. The first and second inclined planes 112 and 114 formed at the substrate 110 may make an excitation light, which is incident at a predetermined angle, incident onto the metal layer 120 at a Surface Plasmon Resonance (SPR) angle. This will be described below in more detail with reference to FIG. 4.

The metal layer 120 is formed along the wedge shape of the upper surface of the substrate 110. In a surface of the metal layer 120, a surface plasmon is generated by external electromagnetic wave (i.e., energy or wavelength). For example, the metal layer 120 may be formed of gold (Au), silver (Ag), chromium (Cr), nickel (Ni) or titanium (Ti).

Moreover, an adhesive layer (not shown) for enhancing the adhesive strength of the metal layer 120 may be formed at an interface of the substrate 110 and the metal layer 120. As the adhesive layer (not shown), for example, a Cr thin film or a Ti thin film may be used, and may be formed to the thickness of about 1 nm to about 5 nm.

The dielectric layer 130 for enhancing the transfer efficiency of SPR energy to a fluorescent substance 148, which is fixed by specifically binding between the capture molecules 142 and the target molecules 144, is formed on the metal layer 120. The dielectric layer 130, for example, may be formed of SiO₂, Si₃N₄, TiO₂, Ta₂O₅ or Al₂O₃.

The fluorescent substances 148 may be separated from the metal layer 120 at certain intervals, and thus, when the fluorescent substances 148 are disposed within an effective transfer distance, the transfer efficiency of SPR energy may be improved. The effective transfer distance represents the energy field of a surface plasmon that is scattered at the metal layer 120 when surface plasmon resonance occurs in the metal layer 120. Specifically, when the effective transfer distance from the metal layer 120 to the fluorescent substance 148 is in about 2 nm to about 20 nm, the transfer energy of SPR energy can be maximized. Accordingly, the dielectric layer 130 having a predetermined thickness may be formed so that the fluorescent substances 148 may be disposed within the effective transfer distance between the fluorescent substances 148 and the metal layer 120.

Moreover, the capture molecules 142 may be immobilized at the surface of the dielectric layer 130. Furthermore, the surface of the dielectric layer 130 may be surface-treated to more tightly immobilize the capture molecules 142. For example, a polymer including poly lysine may be formed at the surface of the dielectric layer 130, and a Self-Assembled Monolayer (SAM) may be formed at the surface of the dielectric layer 130.

Moreover, an active group may be derived to the surface of the dielectric layer 130. For example, active groups such as carboxyl group (—COOH), thiol group (—SH), hydroxyl group (—OH), silane group, amine group or epoxy group may be derived to the surface of the dielectric layer 130.

The capture molecules 142 that specifically bind to the target molecules 144 to be analyzed are immobilized at the surface of the dielectric layer 130. In FIG. 1, the capture molecules 142 are immobilized only at an upper portion of the first inclined plane 112 of the substrate 110. However, the capture molecules 142 are also immobilized at an upper portion of the second inclined plane 114 of the substrate 110, in addition to the upper portion of the first inclined plane 112.

As a method for immobilizing the capture molecules 142 at the surface of the dielectric layer 130, chemical adsorption, covalent-binding, electrostatic attraction, co-polymerization or avidin-biotin affinity system may be used.

The capture molecules 142, for example, may be protein, cell, virus, nucleic acid, organic molecules or inorganic molecules. In the case of protein, the capture molecules 142 may be all biomaterials such as antigen, antibody, matrix protein, enzyme and coenzyme. In the case of nucleic acid, the capture molecules 142 may be DNA, RNA, PNA, LNA or a hybrid thereof. Specifically, in an embodiment of the present invention, the capture molecules 142 may be capture antibodies that may specifically bind to antigens.

The target molecules 144 (i.e., antigens) to be analyzed may be specifically bound to the capture molecules 142. At this point, the target molecules 144 may be marked by the fluorescent substance 148 and thereby may be specifically bound to the capture molecules 142. Specifically, detection molecules 146 in which the fluorescent substance 148 is marked specifically binds to the target molecules 144, and thus the target molecules 144 may be marked with the fluorescent substance 148. At this point, the detection molecules 146 and the capture molecules 142 specifically bind to the target molecules 144 in different sites. In an embodiment of the present invention, the detection molecules 146 may be a detection antibody that may specifically bind to an antigen.

In this way, in the biochip according to an embodiment of the present invention, the metal layer 120 and the dielectric layer 130 are formed on the surface of the substrate 110, i.e., the first and second inclined planes 112 and 114. For the analysis of biomaterials, moreover, the binding structure of capture molecules 142-target molecules 144-detection molecules 146-fluorescent substance 148 may be formed on the dielectric layer 130.

The biochip included in the biomaterial detection apparatus according to an embodiment of the present invention may be applied to a DNA chip, a protein chip, a micro army, and a microfluidic chip.

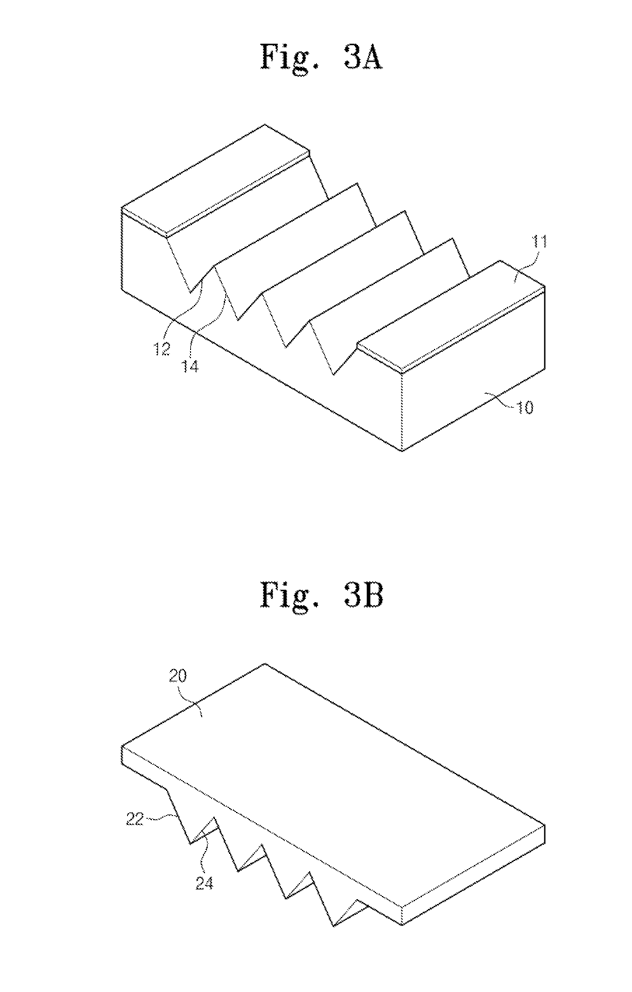

FIG. 2 is a diagram illustrating a biochip according to another embodiment of the present invention.

Referring to FIG. 2, a biochip according to another embodiment of the present invention includes a microfluidic channel 100′. That is, the biochip includes a lower plate 110a and an upper plate (not shown). The lower plate 110a and the upper plate (not shown) are separated from each other at certain interval (for example, channel depth ‘h’) and then are coupled, thereby forming the microfluidic channel 100′. That is, by recessing the certain region of the lower plate 110a from an upper surface to a certain depth ‘h’, the microfluidic channel 100′ may be formed. An upper plate (not shown) may be joined to the upper surface of the lower plate 110a. In the microfluidic channel 100′, a fluid including target molecules may be moved by a capillary phenomenon.

A certain region in which biomaterials react is formed in a wedge shape at the surface of the microfluidic channel 100′ that is formed at the lower plate 110a. That is, the surface of the lower plate 110a includes acute parts 116 that are formed by first and second inclined planes 112 and 114. A plurality of acute parts 116 may be formed at the surface of the lower plate 110a. The first and second inclined planes 112 and 114 formed at the lower plate 110a may make an excitation light, which is incident to the lower plate 110a at a predetermined angle, incident onto a metal layer 120 (See FIG. 1) at an SPR angle.

The acute parts 116 formed at the lower plate 110a may change the distance between the lower plate 110a and the upper plate (not shown). In other words, the microfluidic channel 100′ includes a region in which the distance between the lower plate 110a and the upper plate (not shown) is maintained at ‘h’, and a region in which the distance between the lower plate 110a and the upper plate (not shown) becomes narrower than ‘h’. Accordingly, when the fluid including the target molecules is provided to the microfluidic channel 100′, the providing speed of the fluid may be controlled.

Moreover, the metal layer 120 (see FIG. 1) and a dielectric layer 130 (see FIG. 1) are sequentially formed on the first and second inclined planes 112 and 114 that are formed at the lower plate 110a. Capture molecules 142 for detecting target molecules 144 are immobilized at the surface of the dielectric layer 130 (see FIG. 1).

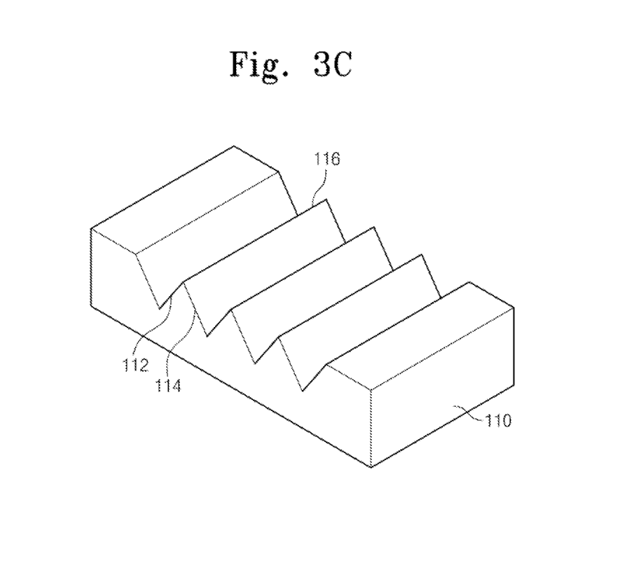

FIGS. 3A through 3C are diagrams illustrating a method of fabricating a biochip according to an embodiment of the present invention.

A substrate, having a wedge-shape upper surface at a certain region, may be formed through photolithography, electronic beam lithography or imprint technology.

To provide a detailed description, as illustrated in FIG. 3A, a single crystal silicon substrate 10 is prepared, a mask 11 which exposes the certain region for forming the wedge-shape upper surface is formed. By performing an anisotropic wet etching process for first and second inclined planes 12 and 14 to be formed at the silicon substrate 10, a groove may be formed at the silicon substrate 10. For example, by etching the silicon substrate 10 with KOH solution, an angle between the first and second inclined planes 12 and 14 may be formed at about 55 degrees (particularly, etching angle 54.7 degrees), at the temperature of about 80° C.

Referring to FIG. 3B, the silicon substrate 10 having a wedge shape of groove is filled with metal materials through an electroplating process, and a metal stamp 20 may be formed by separating the silicon substrate 10 and a metal layer. Consequently, wedge-shape grooves formed at the silicon substrate 10 may be transferred to a surface of the metal stamp 20. Accordingly, first and second inclined planes 22 and 24 that form a predetermined angle at the surface of the metal stamp 20 may be formed. At this point, the metal stamp 20 may use a Ni/Cr thin film or a Ni/Au thin film.

Referring to FIG. 3C, a substrate 100 for forming a biochip is prepared. The substrate 110 may be a plastic or polymer substrate. First and second inclined planes 112 and 114 are formed at a certain region of the substrate 110 with the metal stamp 20. That is, by extrusion-molding or injection-molding the plastic substrate with the metal stamp 20, the substrate 110 having a wedge-shape upper surface may be formed.

FIG. 4 is a diagram illustrating a biomaterial detection apparatus according to an embodiment of the present invention.

Referring to FIG. 4, a biomaterial detection apparatus according to an embodiment of the present invention includes a biochip 100, a light source unit 200 and a detection unit 300.

The biochip 100, as described above with reference to FIG. 1, includes a substrate 110 in which the certain region of an upper surface is formed in a wedge shape, a metal layer 120, a dielectric layer 130, and capture molecules 142.

Capture molecules 142 are immobilized to upper portions of first and second inclined planes 112 and 114 of the substrate 110, and target molecules 144 marked with a fluorescent substance 148 are specifically bound to the capture molecules 142.

Surface plasmon resonance may occur by an excitation light which is incident at a specific angle, in the metal layer 120 that is formed on the first and second inclined planes 112 and 114 of the substrate 110.

Specifically, surface plasmon resonance denotes the oscillation of quantized electrons that occurs because electrons existing inside the metal layer 120 are polarized when light having specific wavelength is irradiated onto the surface of the metal layer 120.

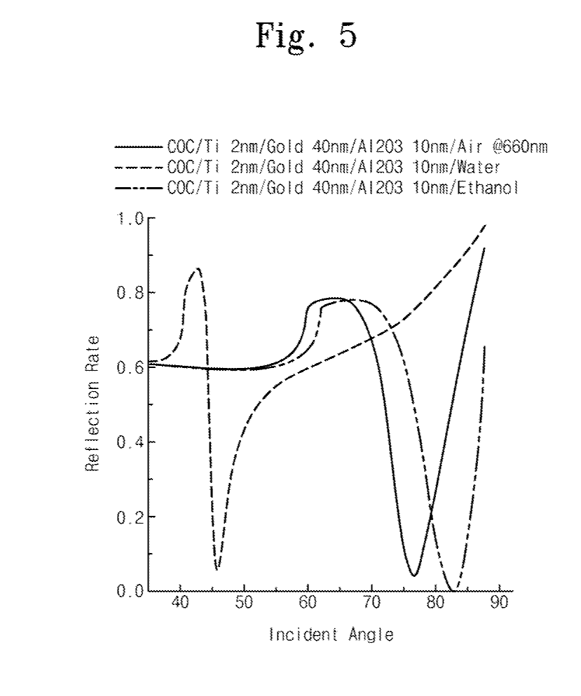

Moreover, when light having specific wavelength is incident to the surface of the metal layer 120 at the specific angle, light is absorbed and scattered by the metal layer 120 and thereby surface plasmon resonance in which the plasmon of the surface of the metal layer 120 is excited may occur. To provide a detailed description, when light is incident at a specific incident angle (for example, SPR angle ‘Θ_{R}’), the wave and phase of a surface plasmon that is generated at the boundary between the metal layer 120 and the dielectric layer 130 are matched, and thus all the energy of the excitation light incident to the metal layer 120 is absorbed to the metal layer 120 and then a reflection wave is eliminated. That is, light having a specific wavelength is absorbed in the surface of the metal layer 120, and light having the specific wavelength is scattered according to materials surrounding the surface of the metal layer 120. This will be described below with reference to FIG. 5.

In this way, the SPR angle is an angle in which the reflection rate of the excitation light incident to the metal layer 120 is rapidly reduced, and it is changed according to the ambient materials of the metal layer 120. This will be described below with reference to FIG. 5.

Accordingly, the excitation light should be incident to the metal layer 120 at the specific angle, for causing surface plasmon resonance at the metal layer 120. Then, when the SPR angle is relatively large, it may be difficult to irradiate the excitation light onto the metal layer 120 at the SPR angle. On the other hand, in an embodiment of the present invention, the metal layer 120 is formed on the first and second inclined planes 112 and 114 of the substrate 110 having the SPR angle, even the excitation light of a small incident angle “90-Θ” for a flat substrate may be incident to the metal layer 120 at the SPR angle ‘Θ_{R}’.

Moreover, when the excitation light is incident to the metal layer 120 at the SPR angle ‘Θ_{R}’, a surface plasmon that is excited at the surface of the metal layer 120 has energy and is scattered, and thus, resonance energy radiated at the metal layer 120 may be transferred to the fluorescent substance 148 that is immobilized by specifically binding between the capture molecules 142 and target molecules 144 of the an upper portion of the metal layer 120.

The light source unit 200 irradiates the excitation light onto the metal layer 120 that is formed on the substrate 110 having a wedge shape. At this point, the light source unit 200 irradiates the excitation light ‘L_{EX}’ at a specific incident angle “90-Θ” for the lower surface of the flat substrate 110. The excitation light ‘L_{EX}’ may be incident to the metal layer 120 at the SPR angle ‘Θ_{R}’ in the first inclined plane 112 or the second inclined plane 114.

That is, although the excitation light ‘L_{EX}’ irradiated in the light source 210 is not irradiated at a specific angle that causes surface plasmon resonance, it may be incident to the metal layer 120 at the SPR angle ‘Θ_{R}’ by the first inclined plane 112 or the second inclined plane 114 of the metal layer 120. Accordingly, a surface plasmon may be excited at the surface of the metal layer 120.

As the light source unit 200, a xenon lamp for outputting polychromatic light may be used. When using the xenon lamp as a light source, the light source unit 200 may provide monochromatic light as the excitation light, including an optical filter. As the light source unit 200, moreover, a white light source, a laser diode or a light emitting diode (LED) may be used.

The detection unit 300 detects a fluorescent signal ‘L_{EM}’ (i.e., emitted light) that is radiated from the fluorescent substance 148 which is immobilized to the upper portions of the first and second inclined planes 112 and 114. At this point, the fluorescent signal ‘L_{EM}’ (i.e., emitted light) that is radiated from the fluorescent substance 148 may be radiated by receiving the resonance energy of a surface plasmon that is excited at the surface of the metal layer 120.

FIG. 5 is a graph illustrating the change of a reflection rate based on an incident angle of an excitation light.

In the graph of FIG. 5, ambient materials have been provided to a microfluidic channel including a metal layer and a dielectric layer, and the change of a reflection rate based on an incident angle of light incident to the metal layer has been detected. As ambient materials provided to the microfluidic channel, an air layer, water and ethanol have been used. Among these, the air layer denotes that the microfluidic channel has been dried. Herein, an incident light has used monochromatic light of about 660 nm that is linearly polarized.

Referring to FIG. 5, the change of a reflection rate in the metal layer based on the incident angle of an excitation light can be known for each of the ambient materials on the metal layer. That is, it can be seen that the reflection rate is rapidly reduced in a specific incident angle for each dielectric layer which exists at a surface of the metal layer. In other words, the graph of FIG. 5 denotes that light incident to the metal layer is resonance absorbed in a specific angle. An SPR angle is an angle when the reflection rate is rapidly reduced in the metal layer. Referring to FIG. 5, moreover, it can be seen that the SPR angle is changed according to materials contacting the surface of the metal layer.

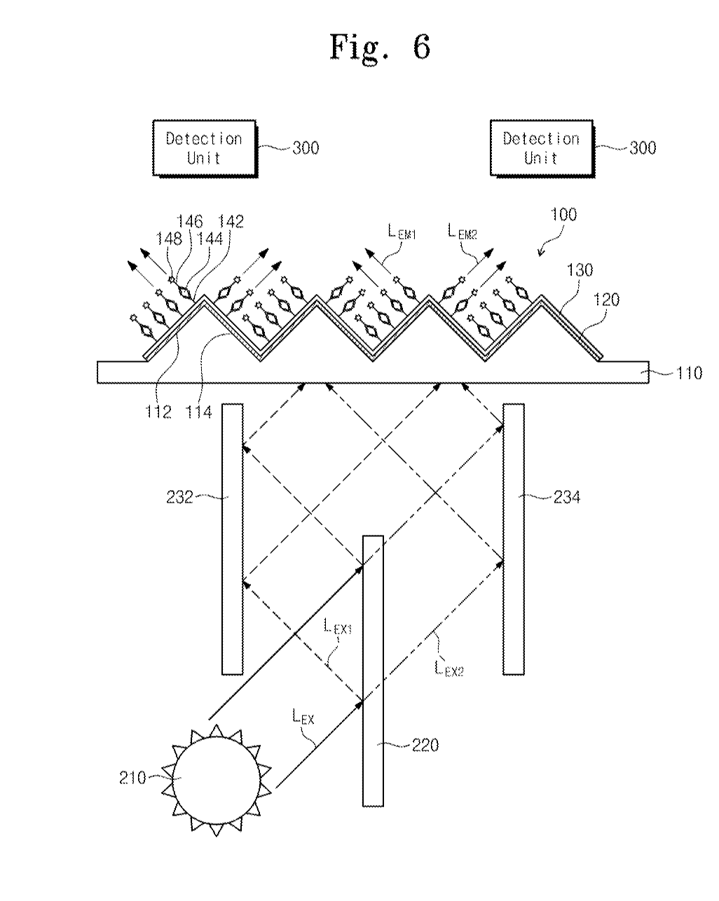

FIG. 6 is a diagram illustrating a biomaterial detection apparatus according to another embodiment of the present invention.

Referring to FIG. 6, a biomaterial detection apparatus according to another embodiment of the present invention enables to detect fluorescent signals ‘L_{EM1} and L_{EM2}’ that are radiated from surfaces of first and second inclined planes 112 and 114 of a substrate 110 by an excitation light ‘L_{EX}’ which is incident at a specific angle.

To provide a detailed description, in another embodiment of the present invention, a light source unit includes a light source 210, a beam splitter 220, a first reflection mirror 232 and a second reflection mirror 234.

That is, the light source 210 irradiates the excitation light ‘L_{EX}’ having a specific wavelength at a certain incident angle. The excitation light ‘L_{EX}’ irradiated at the certain incident angle is transmitted and reflected by the beam splitter 220, and thereby, may be divided into a first excitation light ‘L_{EX1}’ and a second excitation light ‘L_{EX2}’. The first excitation light ‘L_{EX1}’ is provided to the first reflection mirror 232, and may be incident to the first inclined plane 112 of the substrate 110 by being reflected through the first reflection mirror 232. Moreover, the second excitation light ‘L_{EX2}’ is provided to the second reflection minor 234, and may be incident to the second inclined plane 114 of the substrate 110 by being reflected through the second reflection mirror 234. That is, the excitation light ‘L_{EX}’ that is incident at the certain incident angle may be divided into the first excitation light ‘L_{EX1}’ and the second excitation light ‘L_{EX2}’, and the first excitation light ‘L_{EX1}’ and the second excitation light ‘L_{EX2}’ may respectively be provided to the first and second inclined planes 112 and 114 at an SPR angle.

Accordingly, the excitation light may be incident to the first and second inclined planes 112 and 114 at the SPR angle. Therefore, surface plasmon resonance may occur by the first excitation light ‘L_{EX1}’ in a metal layer 120 disposed on the first inclined plane 112. Consequently, an SPR energy may be transferred to a fluorescent substance 148 that is immobilized onto the first inclined plane 112 by specifically binding between target molecules 144 and capture molecules 142. Even in the metal layer 120 disposed on the second inclined plane 114, surface plasmon resonance may occur by the second excitation light ‘L_{EX2}’, and consequently, the SPR energy may be transferred to the fluorescent substance 148 that is immobilized on the second inclined plane 114. Accordingly, a detection unit 300 may detect the fluorescent signals ‘L_{EM1} and L_{EM2}’ that are radiated from the fluorescent substances 148 on the first and second inclined planes 112 and 114.

In embodiments of the present invention, moreover, the excitation light incident to the substrate 110 and light emitted from the fluorescent substance 148 are spatially resolved by the substrate 110, and thus, the detection unit 300 can efficiently detect only light emitted from the fluorescent substance 148 even without using an optical filter that passes through only emitted light. Therefore, the signal to noise ratio (SNR) of the fluorescent signal for detecting the target molecules 144 can be improved.

In embodiments of the present invention, the light source unit 200 may irradiate an excitation light of a first wavelength and an excitation light of a second wavelength, i.e., may irradiate a plurality of excitation lights at certain time intervals. Consequently, a point when the excitation light of the first wavelength is incident to the first and second inclined planes 112 and 114 of the substrate 110 may be different from a point when the excitation light of the second wavelength is incident. Therefore, in the first inclined plane 112 and/or the second inclined plane 114, a fluorescent light by the excitation light of the first wavelength and a fluorescent light by the excitation light of the second wavelength can be obtained at different times. Accordingly, the detection unit 300 can resolve and detect the fluorescent signal radiated from the first inclined plane 112 and the fluorescent signal radiated from the second inclined plane 114 with time.

For detecting various kinds of the target molecules 144, the light source 210 may irradiate the excitation light ‘L_{EX}’ of a plurality of wavelengths at a certain incident angle. That is, when using the beam splitter 220 as a dichroic mirror, the excitation light ‘L_{EX}’ irradiated at the certain incident angle is transmitted at a specific wavelength and is reflected at a specific wavelength, and thereby may be divided into the first excitation light ‘L_{EX1}’ and the second excitation light ‘L_{EX2}’. Therefore, the detection unit 300 can spatially resolve and detect the fluorescent signal radiated from the first inclined plane 112 and the fluorescent signal radiated from the second inclined plane 114 that have different light-emitting center wavelengths. As described above, various kinds of the target molecules 144 may be detected in a single channel through a wavelength division scheme or a time division scheme.

According to the biochip and the biomaterial detection apparatus, by forming an upper surface of a substrate on which capture molecules and target molecules specifically bind in a wedge shape, an incident light irradiated at a certain angle to the substrate can be incident to a metal layer at an SPR angle. Accordingly, the biochip and the biomaterial detection apparatus can radiate a fluorescent signal that is excited by a surface plasmon from a fluorescent substance which is fixed to the upper portion of the substrate by specifically binding between the capture molecules and the target molecules.

Moreover, since the substrate has the wedge shape of upper surface, the biochip and the biomaterial detection apparatus can control the providing amount and speed of a fluid when the fluid including the target molecules is provided to the upper surface of the substrate.

Light incident to the substrate having the wedge shape of upper surface and light (i.e., fluorescent signal) radiated from the fluorescent substance are spatially resolved, and thus a signal to noise ratio (SNR) is improved, thereby more enhancing the sensing efficiency of the biomaterials.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A biochip, comprising:
a substrate including a surface which has a plurality of acute parts formed by first and second inclined planes;
a metal layer on at least one of the first and second inclined planes; and
a dielectric layer on the metal layer, in which capture molecules, specifically binding to target molecules which are marked with a fluorescent substance, are immobilized to a surface of the dielectric layer.

2. The biochip of claim 1, wherein:
the substrate further comprises a microfluidic channel recessed from an upper surface of the substrate to a predetermined depth, and
the surface having the acute parts is formed at the microfluidic channel.

3. The biochip of claim 1, wherein the substrate is a silicon substrate, a glass substrate or a plastic substrate.

4. The biochip of claim 1, wherein the metal layer is formed of gold (Au), silver (Ag), chromium (Cr), nickel (Ni), or titanium (Ti).

5. The biochip of claim 1, wherein a thickness of the dielectric layer is an effective transfer distance of surface plasmon resonance energy which is derived in the metal layer by excitation light irradiated onto the metal layer, or is shorter than the effective transfer distance.

6. The biochip of claim 1, wherein the dielectric layer is formed of SiO₂, Si₃N₄, TiO₂, or Al₂O₃.

7. The biochip of claim 1, wherein the dielectric layer comprises a polymer including poly lysine, or a Self-Assembled Monolayer (SAM).

8. The biochip of claim 1, wherein the capture molecules are immobilized by carboxyl group (—COOH), thiol group (—SH), hydroxyl group (—OH), silane group, amine group or epoxy group which is derived to the surface of the dielectric layer.

9. The biochip of claim 1, wherein the capture molecules comprise at least one selected from group consisting of nucleic acid, cell, virus, protein, organic molecules and inorganic molecules.

10. The biochip of claim 9, wherein the nucleic acid comprises at least one selected from group consisting of DNA, RNA, PNA, LNA and a hybrid thereof.

11. The biochip of claim 9, wherein the protein comprises at least one selected from group consisting of an enzyme, a stroma, an antigen, an antibody, a ligand, an aptamer and a receptor.

12. A biomaterial detection apparatus, comprising:
a substrate including a surface which has a plurality of acute parts formed by first and second inclined planes;
a metal layer on at least one of the first and second inclined planes;
a dielectric layer on the metal layer, in which capture molecules, specifically binding to target molecules which are marked with a fluorescent substance, are immobilized to a surface of the dielectric layer;
a light source unit irradiating an excitation light at a predetermined angle for the first or second inclined plane of the substrate; and
a detection unit detecting an emission light which is emitted from the fluorescent substance which is immobilized by specifically binding between the capture molecules and the target molecules, in one of the first and second inclined planes of the substrate.

13. The biomaterial detection apparatus of claim 12, wherein:
the substrate further comprises a microfluidic channel recessed from an upper surface of the substrate to a predetermined depth, and
the surface having the acute parts is formed at the microfluidic channel.

14. The biomaterial detection apparatus of claim 12, wherein a thickness of the dielectric layer is an effective transfer distance of surface plasmon resonance energy which is derived in the metal layer by excitation light irradiated onto the metal layer, or is shorter than the effective transfer distance.

15. The biomaterial detection apparatus of claim 12, wherein the substrate is disposed between the light source unit and the detection unit.

16. The biomaterial detection apparatus of claim 12, wherein the light source unit comprises:
a light source irradiating the excitation light at the predetermined angle for the first or second inclined plane;
a beam splitter transmitting and reflecting the excitation light to divide the excitation light into a first direction and a second direction;
a first reflection mirror providing the excitation light, which is irradiated in the first direction, to the first inclined plane; and
a second reflection mirror providing the excitation light, which is irradiated in the second direction, to the second inclined plane.

17. The biomaterial detection apparatus of claim 16, wherein the detection unit detects the emission light which is emitted from the fluorescent substance on the first inclined plane and the emission light which is emitted from the fluorescent substance on the second inclined plane.

18. The biomaterial detection apparatus of claim 12, wherein:
the light source unit simultaneously irradiates an excitation light of a first wavelength and an excitation light of a second wavelength, and
the detection unit spatially resolves and detects the emission light which is emitted from the first inclined plane and the emission light which is emitted from the second inclined plane.

19. The biomaterial detection apparatus of claim 12, wherein:
the light source unit irradiates various kinds of excitation lights at different times, and
the detection unit resolves and detects the emission light which is emitted from the first inclined plane and the emission light which is emitted from the second inclined plane, with time.
